(19) United States
(12) Patent Application Publication
(10) Pub. No.: US 2023/0346302 A1
(43) Pub. Date: Nov. 2, 2023

(54) Method for OSA Severity Classification Using Recording-based Peripheral Oxygen Saturation Signal

(71) Applicant: National Yang Ming Chiao Tung University, Hsinchu (TW)

(72) Inventors: Sin Horng Chen, Hsinchu (TW); Cheng Yu Yeh, Hsinchu (TW); Chun Cheng Lin, Hsinchu (TW); Shaw Hwa Hwang, Hsinchu (TW); Yuan Fu Liao, Hsinchu (TW); Yih Ru Wang, Hsinchu (TW); Kai Yang Qiu, Hsinchu (TW); You Shuo Chen, Hsinchu (TW); Yao Hsing Chung, Hsinchu (TW); Yen Chun Huang, Hsinchu (TW); Chi Jung Huang, Hsinchu (TW); Li Te Shen, Hsinchu (TW); Bing Chih Yao, Hsinchu (TW); Ning Yun Ku, Hsinchu (TW)

(21) Appl. No.: 17/732,651

(22) Filed: Apr. 29, 2022

(51) Int. Cl. A61B 5/00; A61B 5/145; G16H 50/50; G16H 50/30

(52) U.S. Cl. CPC A61B 5/4818; A61B 5/14542; A61B 5/7267; G16H 50/50; G16H 50/30

(57) ABSTRACT

The present invention provides a method for OSA (Obstructive Sleep Apnea) severity classification by using recording-based Peripheral Oxygen Saturation Signal. The major feature of the present invention emphasizes on using a recording-based Peripheral Oxygen Saturation Signal (SpO₂ signal) as an input, which is different from the deep learning-based prior art of using segment-based signals as an input to a model, and the segment-based signals has only two classification results, i.e. normal or apnea. The present invention provides a method for a model to detect directly four classification results of the OSA Severity

### FIELD OF THE INVENTION

The present invention relates to a method for OSA (Obstructive Sleep Apnea) severity classification, and more particularly of using a recording-based Peripheral Oxygen Saturation Signal (SpO₂ signal) as an input to detect directly four severity classifications of OSA for output.

### BACKGROUND OF THE INVENTION

Referring to FIG. 1, which shows a deep learning technology of the prior art, using segment-based signals as inputs to differentiate, and only provide two recognition models for OSA, i.e. normal or apnea. A recording-based Peripheral Oxygen Saturation Signal (SpO₂ signal) 1 is segmented to K-segmented SpO₂ signals 2, and fed respectively to the recognition model of two-category for OSA severity , and obtains results corresponding to the segmented SpO₂ signal 4 (normal or apnea), then conduct OSA severity evaluation 5 to show outcome 6.

Using an example to describe the OSA severity evaluation in FIG. 1:

Suppose that a person's sleeping time is 8 hours, OSA severity evaluation can conduct throughout the 8 hours, However, 8 hours are not a fixed time, no limitation to the sleeping time, 8 hours is just an example.

Suppose: A recording-based Peripheral Oxygen Saturation Signal (SpO₂ signal) 1 has a time length of 8 hours;

A segmented SpO₂ signal 2 has a time length T of 60 seconds;

Total cut K=480 segmented SpO₂ signals 2 (K*T=28800 seconds=8 hours);

Suppose OSA severity evaluation 5 shows that L=100 segmented SpO₂ signal 2 are apnea;

Therefore apnea-hypopnea index (AHI) is calculated, AHI=L/ (K*T/3600), which means apnea times per hour:

Normal: AHI<5

Mild: 5≤AHI<15

Moderate: 15≤AHI <30

Severe: AHI ≥30

In the above example, AHI=12.5, which means the result is a mild case.

The above method has three disadvantages: the first is that the recognition is very complicated; the second is that the accuracy of the recognition model is influenced by the different time length of the segment-based signals; the third is that the recognition model uses datasets of segment-based signals during training procedures, which has a very complicated labeling work, the cost of manpower and time is considerable.

### SUMMARY OF THE INVENTION

The object of the present invention is to provide a method for OSA (Obstructive Sleep Apnea) severity- classification by using recording-based Peripheral Oxygen Saturation Signal (SpO₂ signal), the contents of the present invention are decribed as below.

Firstly a recognition model of four-category OSA severity is built up.

Acquire SpO₂ signals from public datasets as the training materials to input into the recognition model of four-category OSA severity for training, and achieve a model.

A recording-based whole SpO₂ signal is inputted into the model for directly showing a recognition result of four-category OSA severity (i.e. normal, mild, moderate or severe).

The recording-based whole SpO₂ signal is inputted into the model, after a processing of an input layer, a feature maps extraction layer based on convolutional neural network, a global average pooling layer, a deuce layer and an output layer to obtain the recognition result of four-category OSA severity.

### BRIEF DESCRIPTION OF THE DRAWINGS

FIG. 1 shows schematically the prior art of using segment-based signals as the input to the model, and provide a recognition model of two-category OSA severity.

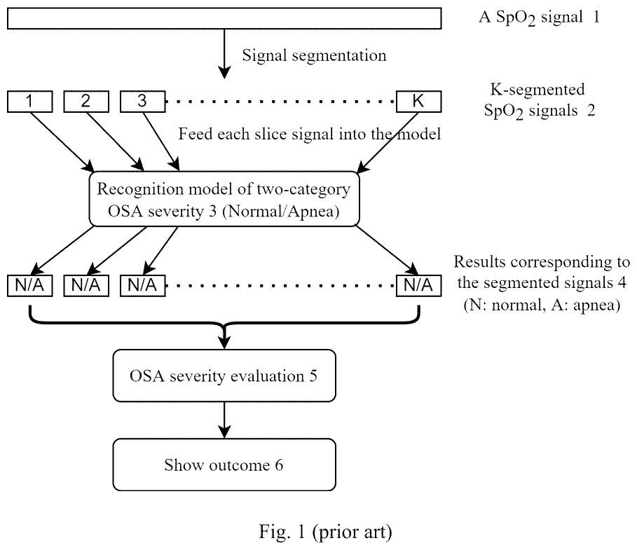

FIG. 2 shows schematically a recording-based whole Peripheral Oxygen Saturation Signal (SpO₂ signal) is used as input for detecting directly and showing a recognition result of four-category OSA severity according to the present invention.

FIG. 3 shows schematically an embodiment of the recognition model of four-category OSA severity in FIG. 2.

FIG. 4 shows schematically the training for generating the recognition model of four-category OSA severity according to the present invention.

FIG. 5 shows schematically a participant wears on the hand a wearable device for measuring SpO₂ and conducting OSA diagnosis according to the present invention.

### DETAILED DESCRIPTIONS OF THE PREFERRED EMBODIMENTS

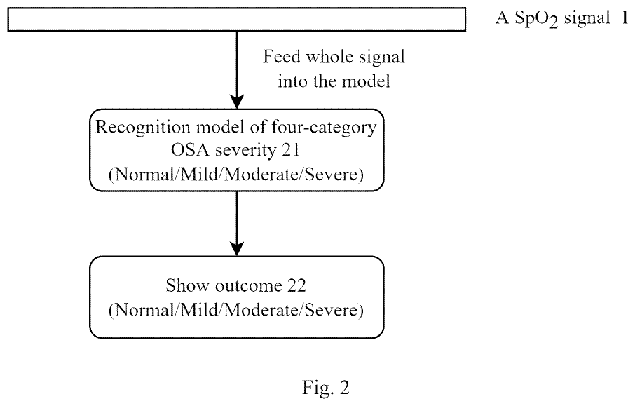

FIG. 2 describes that the present invention uses a recording-based Peripheral Oxygen Saturation Signal (SpO₂ signal) 1 as an input to detect directly four severity classifications of OSA and to show outcome 22 (normal/mild/moderate/severe).

The present invention provides recognition model of four-category OSA severity 21, i.e. normal, mild, moderate or severe, feeds the whole Spa, signal 1 (for example 8 hours, but the time length is not limited) into the model for recognition and shows oucome 22 directly.

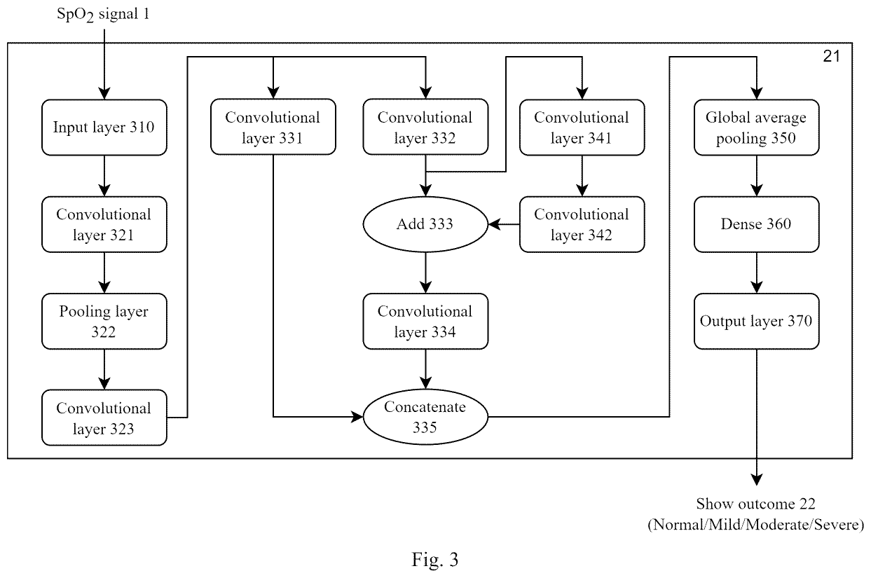

FIG. 3 describes in detail an embodiment of the recognition model of four-category OSA severity 21 in FIG. 2.. The content in FIG. 3 can be divided into five parts, i.e. input layer 310, feature maps extraction layer based on convolutional neural network 321-342, global average pooling 350, deuce 360 and output layer 370.

The input layer 310 : for inputting a whole SpO₂ signal 1. The time length of the input signal according to the present invention is not limited, any time length of the input signal can be used, while the prior art of OSA recognition model (two-category) uses fixed time length for the input signal.

The feature maps extraction layer based on convolutional neural network 321˜342: The feature maps extraction layer uses convolutional neural network (CNN) to conduct feature maps extraction fbr the input signal. The convolutional neural network is used very often in deep learnig. The biggest feature in CNN is to extract automatically feature information of the input signal through model training, which is called feature maps, and then the feature maps is used for conducting recognition. This method can promote the accuracy of recognitionefficiently. The CNN is composed of convolution layers, activation functions and pooling layers. By using these layers to conduct multiple layers of parallel or series connection repeatedly, various CNNs can be built up. In the present embodiment, convolutional layer 321, pooling layer 322 and convolutional layer 323 are the first level for ature aps extraction; convolutional layer 331, convolutional layer 332, add 333, convolutional layer 334 and concatenate 335 are the second level for feature maps extraction, while convolutional layer 341 and. convolutional layer 342 derived from the second level are the third level for feature maps extraction.

The global average pooling 350 A global average pooling method is used for calculate an average value for each feature map as the ouput of the pooling layer. This method can convert input signal of different length into an output signal of the same length. In other word, this method can make the model of the present invention accept input signal of any length.

The deuce 360: to integrate the features of high abstraction obtained above, and then transfer to the output layer 370.

The output layer 370 use softniax activation activation fuction to output a probability value for each category of OSA severity, the sum of the probability value of all categories is 1.

In the present embodiment, all convolutional layers use kernel of 1-dimension, Convolutional layer 321 uses 32 kernels of size 30, expressed as (32, 30), Convolutional layer 323 uses kernels of (64, 3); Convolutional layer 331 uses kernels of (32, 1); Convolutional layer 332 uses kernels of (32, 1); Convolutional layer 334 uses kernels of (32, 1); Convolutional layer 341 uses kernels of (32, 3); Convolutional layer 342 uses kernels of (32, 3).

In the present embodiment, if the input SpO₂ signal 1 to the input layer 310 samples out 28800 sampling points within 8 hours by 1 Hz sampling rate, then after an operation of the convolutional layer 321 to convert into 32 feature maps of size 960. This feature maps is a 2-dimension array, expressed as 960×32. Then the pooling layer 322 uses a sliding window of size 2 to adopt max pooling for the 960×32. feature maps outputted from the convolutional layer 321, so as to obtain a 480×32 feature maps. Thereafter the convolutional layer 323 conducts a convolutional operation to the feature maps outputted from the pooling layer 322, so as to generate a 480×64 feature maps.

Thereafter the convolutional layer 331 and the convolutional layer 332 conduct a convolutional operation respectively to the feature maps outputted from the convolutional layer 323 so as to generate 480×32 feature maps respectively. The convolutional layer 341 conducts a convolutional operation to the feature maps outputted from the convolutional layer 332 so as to generate 480×32 feature maps. The convolutional layer 342 conducts a convolution operation to the feature maps outputted from the convolutional layer 341 so as to generate 480×32 feature caps. Then the feature maps outputted from the convolutional layer 332 and the feature maps outputted from the convolutional layer 342 will conduct addition operation at the add 333 to obtain a merged 480×32 feature maps. The convolutional layer 334 conducts a convolutional operation to the feature maps outputted from the add 333 to obtain 480×32 feature maps. Finally the feature maps outputted from the convolutional layer 331 and the feature maps outputted from the convolutional layer 334 will conduct concatenation process at concatenate 335 to obtain 480×64 feature maps.

The global average pooling 350 in the present embodiment then uses global average pooling method to treat feature maps outputted from the concatenate 335 and obtains 64 feature maps average value. It's worth mentioningthat the global average pooling method can make input of different length to be converted into output of the same length, therefore the input signal of the present invention model can be any length by this method. Therafter the features outputted from the global average pooling 350 will be inputted to the dence 360 having 4 neurons. The output layer 370 then uses soilmax activation function to compute the 4 probability values outputted from the dence 360 to each category of OSA severity. Finally, the show outcome 22 will display an OSA severity category which having the maximal probability value.

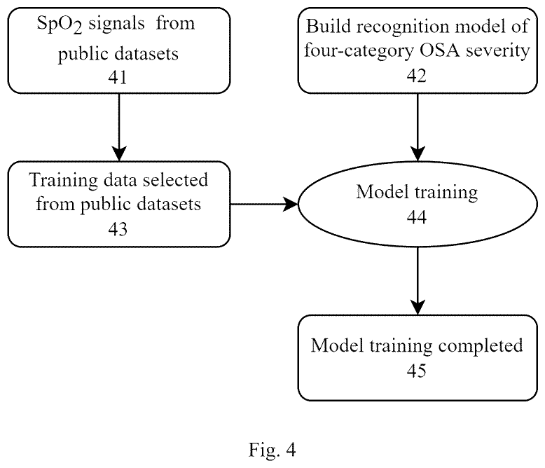

FIG. 4 describes how to train and generate a model according to the present invention. Firstly acquire SpO₂ signals from public datasets 41, build recognition model of four-category OSA severity 42, use Straining data selected from public datasets 43 to input into OSA recognition model for training 44, after model training is completed 45, the model can be nosed to conduct OSA diagnosis.

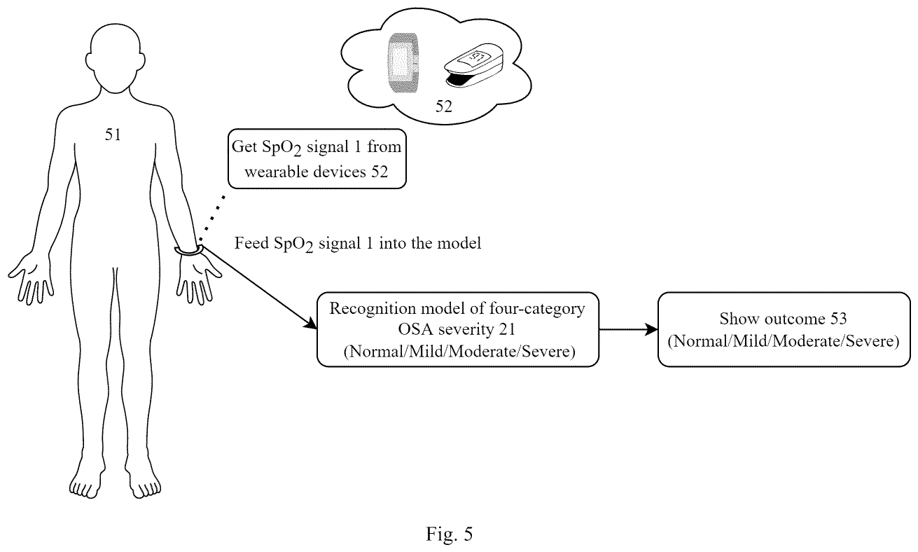

Nowadays wearable devices which can measure Spa, are very popular, it is very convenient to conduct OSA diagnosis through SpO₂ analysis, a user can do self-test at home. Referring to FIG. 5, a participant 51 wears on the hand a wearable device 52 for rrneasuring SpO₂, and obtains SpO₂ signal to input into the recognition model of four-category OSA severity 21 for conducting OSA diagnosis, and show outcome 53 of normal, mild, moderate or severe directly.

The scope of the present invention depends upon the thllowing claims, and is not limited by the above embodiments.

What is claimed is:

1. A method for OSA (Obstructive Sleep Apnea) severity classification by using recording-based Peripheral Oxygen Saturation Signal (SpO₂ signal), comprising:
a. build up a recognition model of four-category OSA severity;
b. acquire SpO₂ signals from public datasets to input into the recognition model of four-category OSA severity for training, and achieve a model;
c. a recording-based whole SpO₂ signal is inputted into the model for showing directly a recognition result of four-category OSA severity (normal, mild, moderate or severe).

2. The method for OSA (Obstructive Sleep Apnea) severity classification by using recording-based Peripheral Oxygen Saturation Signal (SpO₂ signal) according to claim 1, wherein the recording-based whole Spa, signal is inputted into the model, after a processing of an input layer, a feature imps extraction layer based on convolutional neural network, a global average pooling layer, a deuce layer and an output layer to obtain the recognition result of four-category OSA severity.

3. The method for OSA (Obstructive Sleep Apnea) severity classification by using recording-based Peripheral Oxygen Saturation Signal (SpO₂ signal) according to claim 1, wherein a wearable device is used for obtaining the recording-based whole SpO₂ signal.
